(19) United States
(12) Patent Application Publication
(10) Pub. No.: US 2009/0171434 A1
(43) Pub. Date: Jul. 2, 2009

(54) PROSTHESIS LOADING DELIVERY AND DEPLOYMENT APPARATUS

(75) Inventors: Emily Rusk, Boston, MA (US); Peter Shank, Boylston, MA (US); Michael Abi-Kheirs, Weymouth, MA (US)

Correspondence Address: HOFFMANN & BARON, LLP, 6900 JERICHO TURNPIKE, SYOSSET, NY, 11791, US

(73) Assignee: BOSTON SCIENTIFIC SCIMED, INC., Maple Grove, MN (US)

(21) Appl. No.: 12/336,596

(22) Filed: Dec. 17, 2008

Related U.S. Application Data
(60) Provisional application No. 61/017,184, filed on Dec. 28, 2007.

(51) Int. Cl. A61F 2/06; A61B 19/00

(52) U.S. Cl. 623/1.12; 623/1.11; 128/898

(57) ABSTRACT

Prosthesis loading and deploying systems include a capturing device with a proximal stent-engaging member and an elongate pulling member extending distally from the stent-engaging member. With a prosthesis or stent in a relaxed or enlarged-radius state, the pulling member is guided distally through a delivery catheter, pulling the stent-engaging member and prosthesis into the catheter lumen to progressively radially compress the prosthesis to a reduced-radius state. Simultaneously the distal end region of an elongate control device is maintained within a proximal region of the prosthesis, so that the prosthesis is compressed about the control device distal end region as these components enter the catheter. When the prosthesis is compressed about the control device, it tends to follow axial movement of the control device, thus to afford reliable positional control of the prosthesis inside the catheter by manipulating the control device.

### CROSS-REFERENCE TO RELATED APPLICATIONS

This application claims the benefit of U.S. Provisional Application No. 61/017,184, filed Dec. 28, 2007, the content of which is incorporated herein by reference.

### FIELD OF THE INVENTION

The present invention relates to stents, stent-grafts and other intraluminally implantable prostheses, and more particularly to apparatus and methods for loading prostheses into delivery catheters and other prosthesis-confining structures.

### BACKGROUND OF THE INVENTION

A variety of treatment and diagnostic procedures involve devices intraluminally disposed within the body of the patient. Among these devices are stents, including braided stents as disclosed in U.S. Pat. No. 4,655,771 to Wallsten. The Wallsten prostheses or stents are tubular, braided structures formed of helically wound filaments. These stents typically are deployed in a reduced radius state using a delivery catheter including an outer tube. When the stent is positioned at the intended treatment site, the outer tube of the delivery catheter is withdrawn, allowing the stent to radially expand into a substantially conforming surface contact with a blood vessel wall or other lumen-defining tissue.

An alternative stent construction to the braided Wallsten features plastically deformable strands or elements, usually formed of a ductile metal. Examples of such stents are shown in U.S. Pat. Nos. 4,776,337 to Palmaz and 5,716,396 to Williams, Jr. These stents do not require outer tubes or other features to maintain them in the reduced-radius state during delivery. Radial expansion at the treatment site, however, requires a dilatation balloon or other mechanism for radially enlarging the stent.

Regardless of whether the stents are self-expanding or plastically deformable, they generally have an open mesh or open frame construction, or otherwise are formed with multiple openings to facilitate radial enlargements and reductions, and to allow tissue in-growth. Either type of stent may be used to support a substantially fluid-impermeable material, frequently but not necessarily elastic, to provide a stent-graft for shunting blood or other body fluids past a weakened or damaged area such a lesion or stricture.

The structural strands or filaments of braided stents may be formed of metal, typically stainless steel, alloys including cobalt and alloys including titanium. Alternatively, the strands may be polymeric, formed of materials such as polyethylene terephthalate (PET), polypropylene (PP), polyetheretherketone (PEEK), high density polyethylene (HDPE), polysulfone (PSO), polytetrafluoroethylene (PTFE), fluorinated ethylene propylene (FEP), polycarbonate urethane (PC/PU), and polyurethane (PU). As another alternative, the structural strands of stents may be formed of bioabsorbable materials. Metallic stents typically are stronger and more resilient than stents formed of other materials. Nevertheless, there is an increased demand for prostheses formed of polymeric materials or bioabsorbable materials, particularly for use in the treatment of benign diseases and in other situations in which a removable prosthesis or a prosthesis with bioabsorbable structural strands is desirable.

Because of their superior structural strength and resiliency, self-expanding metal prostheses are well suited for preloading into catheters and other prosthesis delivery devices that maintain the prosthesis in a reduced-radius state, facilitating intraluminal delivery of the prosthesis to a designated treatment site. In the case of a radially self-expanding stent or other prosthesis, preloading entails elastically deforming the device to the reduced-radius state and maintaining the device in that state against an internal elastic restoring force of the stent. Upon release of the stent from the delivery device at the designated treatment site, the device radially expands via its restoring force and contacts the surrounding tissue or lumen. Typically, the surrounding tissue or lumen maintains the stent or prosthesis in a slightly radial compressed state, so that the internal restoring force of the stent continues to act radially against the tissue to anchor the device thereat.

Systems with preloaded prostheses are more convenient for the physician and contribute to the success of the procedure. With a prosthesis preloaded into a delivery system, there is no need for the attending physician to radially compress or otherwise manipulate the prosthesis, and his or her attention is more appropriately directed to intraluminal guidance and placement of the prosthesis. A preloaded prosthesis eliminates the time that otherwise would be needed to load a prosthesis, and this is particularly advantageous in time-critical procedures.

Metallic stents, metallic stent-grafts and other metallic prostheses may usually be maintained in their radially-reduced states without experiencing any material reduction in resilience. These devices may be loaded into the delivery system several minutes or several months, or longer, ahead of the deployment procedure. In other words, the duration in the radially compressed state does not materially affect the resilient properties of a metallic stent.

As noted above, implantable prostheses may be formed of polymeric and biodegradable materials, either in total or in part. Certain biodegradable materials, like polymers, may be used to fabricate radially self-expanding stents. In many procedures, polymeric or bioabsorbable prostheses are preferred over metallic devices, for example, due to the relative ease of removing a device intended for temporary implantation, or the capacity to be absorbed into the body.

When maintained in the reduced-radius state under a constant load for any appreciable length of time, a prosthesis formed of polymeric or bioabsorbable material may, however, undergo permanent or plastic deformation. When released from the catheter or other delivery device, such prosthesis may radially self expand to a diameter considerably less than its relaxed-state diameter prior to preloading. This phenomenon is commonly referred to as stress relaxation or “creep”. This phenomenon is aggravated when a polymeric or bioabsorbable prosthesis is exposed to elevated temperatures in its reduced-radius state, for example during a sterilization procedure, which may be performed prior the outset of the prosthesis deployment procedure.

To counteract this phenomenon of stress relaxation or creep, the polymeric or bioabsorbable prosthesis may be sterilized and/or stored in its relaxed state, i.e., not significantly reduced radial state, until just before it is to be used. When the physician is about to begin a procedure, he or she may load the polymeric prosthesis into the delivery system. Consequently, the prosthesis remains compressed in the reduced-radius state only for a short time, perhaps only several minutes. While such a procedure counteracts the problem of creep, the procedure is, however, more difficult and time consuming.

Therefore, it is an object of the present invention to provide a simple and reliable system for loading and deploying a body-insertable and radially expandable prosthesis, in particular one including polymeric material.

Another object is to provide a prosthesis loading and deployment system that affords positive control over the position of the prosthesis, both during its loading and later during its deployment.

A further object is to provide a process for loading a radially expandable prosthesis into a deployment device or other confining structure, with increased ease and simplicity to facilitate loading at the beginning of a deployment procedure.

Another object is to enhance the utility of the inner catheter or member of a prosthesis deployment system.

Yet another object is to provide an apparatus for loading a radially expandable prosthesis into a delivery catheter or other confining structure that reduces the time required for loading, and minimizes the risk of damage to the prosthesis and other components.

### SUMMARY OF THE INVENTION

To achieve these and other objects, there is provided an apparatus for loading a radially expandable prosthesis into a prosthesis confining structure for maintaining the prosthesis in a reduced-radius state, such as a compressed state for a self-expanding stent or prosthesis or a pre-delivery or quiescent state for a balloon-expandable stent or prosthesis. The apparatus includes a prosthesis capturing device with a proximal capturing section that forms a compliant enclosure. The compliant enclosure may be open at a proximal end to allow insertion of a radially expandable prosthesis in an enlarged-radius state, such as relaxed or quiescent state for a self-expanding stent or prosthesis and an enlarged state for a balloon-expandable stent or prosthesis, into the enclosure, whereby the prosthesis is surrounded or at least partially surrounded by the enclosure, including over a distal region of the prosthesis. The capturing device may further include an elongate enclosure moving section, insertable into and movable distally along a lumen or other passage of the prosthesis confining structure to locate the enclosure, and thus also locate the radially expandable prosthesis so surrounded by the enclosure, adjacent a proximal entrance of the passage. The apparatus may further include an elongate control device having a distal end region insertable into a radially expandable prosthesis in the enlarged-radius state. The distal end region may be adapted for a releasable engagement with a radially expandable prosthesis when surrounded by the prosthesis in a reduced-radius state. When in the releasable engagement, the prosthesis may track axial movement of the control device. The enclosure moving section, with the enclosure and a radially expandable prosthesis so located and with the prosthesis surrounding the distal end region of the control device, may be movable distally to draw the enclosure and the prosthesis into the passage to cause a progressive radial compression of the enclosure and prosthesis, radially contracting the prosthesis to the reduced-radius state about the distal end region to effect the releasable engagement.

In one embodiment, the axial length of the enclosure is such that the proximal region of the radially expandable prosthesis, which is selected for insertion into the enclosure, remains outside the enclosure following its insertion. In some embodiments, the proximal region of the selected prosthesis constitutes at least one-half of the prosthesis length. Then, the enclosure moving section is operable to pull the enclosure distally through the lumen of a prosthesis confining structure until the enclosure is free of the lumen, with the proximal region of the prosthesis remaining in the lumen, still releasably engaged with the control device. This may eliminate the need to pull the proximal capturing section out from between the prosthesis and the confining structure. In some embodiments, the capturing device does not pull the prosthesis as it is removed, as there is less need to pin down the prosthesis at the distal end and less need to push an exposed prosthesis distal end into the confining structure to complete the loading function. Accordingly, several causes of prosthesis damage in prior loading devices may be eliminated.

The proximal capturing section can comprise an open mesh or open weave stent-engaging member. In one approach, the stent-engaging member is formed of helically wound, interbraided strands. This embodiment is well suited for use with a prosthesis formed of helical interbraided strands, because both the stent-engaging member and the prosthesis tend to elongate axially as they are radially compressed. Also, the respective braids have a tendency to engage one another, which may improve the capacity of the stent-engaging member to hold the prosthesis as the stent-engaging member and the prosthesis are drawn into the delivery catheter or other confining structure.

To provide the desired prosthesis retention quality, the control device may be constructed with a low durometer material or formed with a high friction surface along its distal tip. A prosthesis holding feature may be provided in the form of a prosthesis holding sleeve surrounding the control device, or several strips mounted to the control device. When used with open weave, open mesh or braided prostheses, the holding sleeve may comprise a compliant, low durometer material that tends to conform to the prosthesis as the prosthesis is compressed around it in the reduced-radius state. This helps to ensure that, when the control device and the outer catheter or other confining structures are moved axially relative to one another, the prosthesis follows the control device rather than the confining structure.

The holding feature can be mounted at the distal end of the control device. Alternatively, the holding feature can be proximally spaced apart from the control device distal end. This arrangement may be advantageous when the control device incorporates a catheter balloon, inflatable to radially expand either a ductile, e.g., plastically deformable, stent or a self-expanding stent at the intended treatment site. Typically, the balloon extends from a point near the distal end to a point just distally of the holding feature with the radially compressed stent overlying the balloon and feature.

In one version of the apparatus, the confining structure comprises a prosthesis delivery catheter incorporating an axially extended catheter lumen, and the control device comprises an inner member contained in the lumen and movable axially relative to the catheter to deploy a radially expandable prosthesis at a selected treatment site.

In other versions, the confining structure may be an intermediate device, for example either a loading tube or a loading capsule. The loading tube may have a diameter substantially the same as the diameter of a delivery catheter, whereby the tube is positionable to abut the catheter distal end to accommodate transfer of the prosthesis from the tube to the catheter. The tube is removable from the catheter, leaving the prosthesis and control device contained therein.

Alternatively a loading capsule, at least near its proximal end, may have a lumen substantially equal in diameter to a delivery catheter lumen. The capsule may be positionable with its proximal end in confronting relation to the distal end of a delivery catheter, to accommodate a proximal transfer of the prosthesis from the capsule to the catheter. This version can advantageously employ a socket designed to establish the confronting relation while releasably maintaining the capsule and catheter coaxially aligned. If desired, the capsule lumen can have a larger diameter over most of its length, necked down to equal the catheter lumen diameter at the capsule proximal end. This may facilitate the use of the capturing device to load the prosthesis into the capsule.

Another aspect of the present invention is a package or assembly including the prosthesis capturing device, a radially expandable prosthesis in the enlarged-radius state surrounded by the capturing device enclosure, and/or the elongate control device. If desired, the assembly or package further can include a prosthesis delivery catheter or other confining structure. In addition, the package can incorporate a tray or other support for maintaining the various components in a desired configuration, particularly with the prosthesis contained in the enclosure, and optionally with the control device distal end surrounded by the prosthesis. The assembly may also provide a convenient vehicle for simultaneously sterilizing these components, and for transporting and otherwise handling these components both before and after the sterilization stage.

In further embodiments, a stent loading and deploying device may include a stent confining device for maintaining a radially expandable stent in a reduced-radius state suitable for delivering the stent to an intraluminal treatment site. The device may have a stent capturing device including a proximal capturing section forming a compliant enclosure open at a proximal end to allow insertion of the stent, when in an enlarged-radius state, into the enclosure to be surrounded by the enclosure along a distal region of the stent. The capturing device may further include an elongate enclosure moving section insertable into and movable distally through a passage running axially along the stent confining device, to locate the enclosure, and the stent when so contained in the enclosure, adjacent a proximal entrance of the passage. The deployment device may further include an elongate stent control device having a distal end region insertable into the stent when the stent is in the enlarged-radius state. The distal end region may be adapted for a releasable engagement with the stent when surrounded by the stent with the stent in the reduced-radius state, whereby the stent tends to follow axial movement of the control device. The moving section, with the enclosure and stent so located and with the stent surrounding the distal end region, may be movable distally to draw the enclosure and stent into the passage and to progressively radially compress the enclosure and stent, thereby radially contracting the stent to the reduced-radius state about the distal end region to effect the releasable engagement.

Further in accordance with the invention, there may be provided a process for loading a radially expandable stent into a confining structure for maintaining the stent in a reduced-radius state, comprising the following steps:
(a) providing a radially expandable stent in an enlarged-radius state, and a compliant enclosure surrounding a distal region of the stent;
(b) providing an elongate control device having a distal end region;
(c) inserting the control device into the stent to position a proximal region of the stent in surrounding relation to the control device distal end region; and
(d) with the stent maintained in the surrounding relation to the control device distal end region, drawing the enclosure and the stent surrounded by the enclosure distally into a lumen of a stent confining structure to cause a progressive radial contraction of the enclosure and stent as they enter the lumen, to contract the stent to a reduced-radius state about the control device distal end region, thereby to effect a releasable engagement of the stent with the control device whereby the stent tends to track axial movement of the control device.

The process further can include providing a retaining feature along the control device distal end region. Then, the stent may be maintained in surrounding relation to the retaining feature, with the releasable engagement comprising engagement of the stent directly with the retaining feature.

The stent may have a proximal region extending away from the enclosure when captured in the enclosure. This proximal region may be aligned with the retaining feature. Then, after the enclosure and stent are drawn into the confining device, the enclosure and stent may be movable further distally until the enclosure is free of the confining device, while the proximal region of the stent remains in the passage, releasably engaged with the retaining feature. This may facilitate removal of the enclosure from the confining structure and may permit the pulling of the control device proximally to draw the stent completely back into the passage after the enclosure is removed. When the confining device is a delivery catheter, this may position the stent for intraluminal delivery to an intended treatment site.

In accordance with a further aspect of the invention, the control device can incorporate a releasable coupling between the distal end region comprising a distal tip section incorporating the retaining feature, and a proximal region or section that typically forms most of the control device axial length. For example, the connection can employ complementary threads, a pin in one of the section insertable into a groove in the other, or a snap fit. In any event, the coupling may permit a stent or prosthesis to be loaded into an intermediate confining structure such as a loading tube or loading capsule, using just the distal tip section of the control device. The tip section may be relatively short, e.g. from about 1 cm to 10 cm, desirably from about 1 cm to about 5 cm, including less than 3 cm, and is much easier to manipulate during loading than the complete control device, which can have a length of 80 cm or more, for example from about 80 cm to about 300 cm, including from about 80 cm to about 200 cm. The proximal section can be loaded into a delivery catheter and coupled to the assembly that includes the distal tip section, the stent, and the loading tube or capsule containing them. Then, the assembled control device can be used to transfer the stent to the delivery catheter as previously described.

A variety of distal tip sections, for example a tip section carrying only a holding sleeve and a tip section including a balloon catheter as well, can be provided for use with a single control device proximal section. Further, a variety of distal tips representing different procedures may be used with the proximal section. For example, a control device proximal section might be used with a dilating distal tip to enlarge a lumen at an intended treatment site, then with a different distal tip for loading and deploying a stent to the treatment site, and finally with a balloon distal tip to radially enlarge a stent after its implantation for a more secure fixation.

Thus in various embodiments, radially expandable stents and other prostheses can be loaded into delivery catheters and other confining structures with relative ease and simplicity, immediately before an implantation procedure. This allows the physician to select a device most suitable for the procedure at hand, even when the device is subject to creep or otherwise not suitable for long term maintenance in a reduced-radius state. The present system not only may reduce the time required for on-site loading, but may also minimize the risk of damage to the prosthesis and other components, by allowing manipulation of the prosthesis without pushing against or crimping one of its ends. Further, the same control device used to position the prosthesis during loading, may also be used to control the prosthesis relative to a delivery catheter during deployment. Device loading can be further simplified by intermediate confining devices such as loading tubes and capsules, and by forming the control device with a distal tip section removably coupled to the remainder of the device. In both cases, the user is able to manipulate and load relatively short components, in lieu of the much longer delivery catheter and inner member needed during deployment.

### BRIEF DESCRIPTION OF THE DRAWINGS

For a further understanding of the above objects and advantages, reference is made to the following detailed description and to the drawings, in which:

FIG. 1 is an elevational view, partially in section, of one embodiment of a stent loading and deploying system constructed in accordance with the invention;

FIG. 2 is an enlarged partial view of one embodiment of a stent capturing device of the loading and deploying system;

FIG. 3 is an enlarged partial view of one embodiment of a stent control device of the loading and deploying system;

FIG. 4 is a partial elevational view of one embodiment of the system, with components positioned for stent loading;

FIGS. 5-9 schematically illustrate same embodiments of a stent loading sequence;

FIGS. 10-11 schematically illustrate same embodiments of a stent deployment sequence;

FIG. 12 illustrates an alternative embodiment in the form of a stent loading system;

FIGS. 13-15 illustrate a stent loading sequence using the system of FIG. 12;

FIG. 16 illustrates an alternative embodiment stent loading system;

FIGS. 17-23 illustrate one embodiment of a stent loading sequence using the system of FIG. 16;

FIG. 24 shows an alignment socket usable with the system of FIG. 16;

FIG. 25 illustrates an alternative embodiment control device with a detachable distal tip;

FIGS. 26 and 27 illustrate alternative embodiment detachable distal tips;

FIG. 28 illustrates an alternative embodiment control device with a detachable tissue dilating tip;

FIG. 29 illustrates a further alternative embodiment control device including a detachable dilating tip with a dilatation balloon;

FIGS. 30 and 31 illustrate alternative embodiment control devices with alternative stent retaining features;

FIG. 32 illustrates alternative stent capturing section of a capturing device; and

FIG. 33 illustrates one embodiment of a packaging assembly for a stent and for certain loading and/or delivery components.

### DETAILED DESCRIPTION OF THE PREFERRED EMBODIMENTS

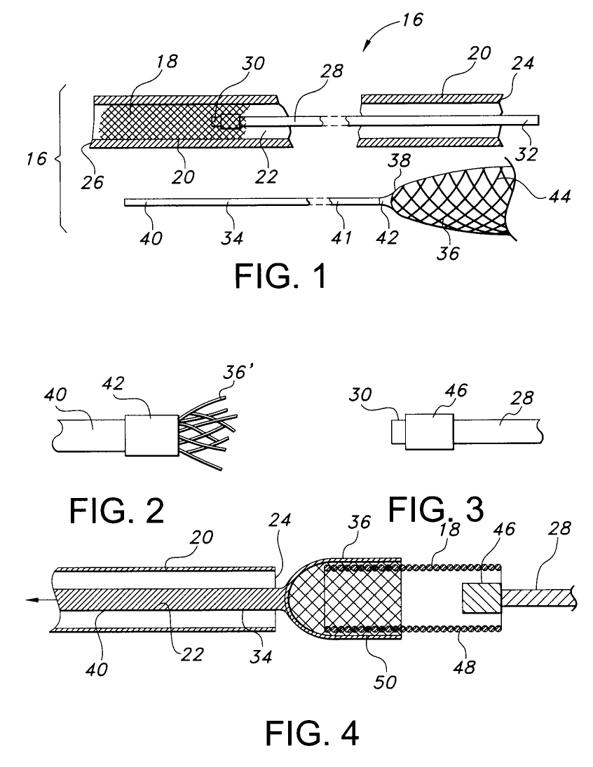

Turning now to the drawings, there is shown in FIG. 1 a stent loading and deploying system 16 for loading and deploying a radially self-expanding stent 18. While system 16 and alternative embodiment systems are discussed primarily in connection with deploying radially self-expanding stents, it can be appreciated that these systems may be used to deploy other implantable devices as well, e.g. balloon-expandable stents, grafts, and stent-grafts. As used herein, the use of the term stent may refer to and/or comprise any implantable prosthesis for use in a body lumen.

System 16 employs several components, some of which are involved in loading stent 18 and others are involved in stent deployment. The later components include an elongate, pliable outer catheter or tubing 20 constructed of a biocompatible polymer. Suitable polymers include, but are not limited to, polytetrafluoroethylene (PTFE), polypropylene (PP), or polyethylene terephthalate (PET). A central lumen 22 runs axially through catheter 20, from a proximal end 24 to a distal end 26 of the catheter 20. During a deployment procedure, catheter 20 may be inserted by distal end 26 and is then guided intraluminally to a selected treatment site, while proximal end 24 remains outside the body.

The outer catheter 20 is shown in section to reveal stent 18 and an elongate stent control device or inner member 28. The control device 28 may be formed of a biocompatible polymer such as, but not limited to, PTFE, PP, PET or polyamide (PA), commonly referred to as nylon. Control device 28 is flexible and pliable to allow bending when negotiating body lumens, including but not limited to blood vessels, and also has sufficient axial stability to permit a physician to control the position of distal end 26 by manipulating proximal end 24.

Stent 18 may be of open weave or mesh construction and, in some embodiments, may be formed of multiple interbraided helically wound strands or filaments. Stent 18, however, is not limited to a braided stent and other stent configurations may suitably be used. Useful biocompatible materials include but are not limited to biocompatible metals, biocompatible alloys, biocompatible polymeric materials, including synthetic biocompatible polymeric materials and bioabsorbable or biodegradable polymeric materials, materials made from or derived from natural sources and combinations thereof. Useful biocompatible metals or alloys include, but not limited to, nitinol, stainless steel, cobalt-based alloy such as Elgiloy, platinum, gold, titanium, tantalum, niobium, polymeric materials and combinations thereof. Useful synthetic biocompatible polymeric materials include, but are not limited to, polyesters, including polyethylene terephthalate (PET) polyesters, polypropylenes, polyethylenes, polyurethanes, polyolefins, polyvinyls, polymethylacetates, polyamides, naphthalane dicarboxylene derivatives, silks and polytetrafluoroethylenes. The polymeric materials may further include a metallic, a glass, ceramic or carbon constituent or fiber. Useful and nonlimiting examples of bioabsorbable or biodegradable polymeric materials include poly(L-lactide) (PLLA), poly(D,L-lactide) (PLA), poly(glycolide) (PGA), poly(L-lactide-co-D,L-lactide) (PLLA/PLA), poly(L-lactide-co-glycolide) (PLLA/PGA), poly(D,L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D,L-lactide-co-caprolactone) PLA/PCL), poly(glycolide-co-caprolactone) (PGA/PCL), poly(phosphate ester) and the like, and any one of the materials identified in U.S. Pat. No. 6,245,103, the contents of which are incorporated herein by reference by their entirety. Further, the stent 18 may include materials made from or derived from natural sources, such as, but not limited to collagen, elastin, glycosaminoglycan, fibronectin and laminin, keratin, alginate, combinations thereof and the like. Further, the stent 18 may be made from polymeric materials which may also include radiopaque materials, such as metallic-based powders or ceramic-based powders, particulates or pastes which may be incorporated into the polymeric material. For example, the radiopaque material may be blended with the polymer composition from which the polymeric filament is formed, and subsequently fashioned into the stent as described herein. Alternatively, the radiopaque material may be applied to the surface of the metal or polymer stent. Various radiopaque materials and their salts and derivatives may be used including, without limitation, bismuth, barium and its salts such as barium sulfate, tantalum, tungsten, gold, platinum and titanium, to name a few. Additional useful radiopaque materials may be found in U.S. Pat. No. 6,626,936, which is herein incorporated in its entirety by reference. Metallic complexes useful as radiopaque materials are also contemplated. The stent 18 may be selectively made radiopaque at desired areas along the stent 18 or made be fully radiopaque, depending on the desired end-product and application. Further, portions of the stent 18, for example stent filaments, may have an inner core of tantalum, gold, platinum, iridium or combination of thereof and an outer member or layer of nitinol to provide a composite filament for improved radiocapicity or visibility. Alternatively, the stent 18 may also have improved external imaging under magnetic resonance imaging (MRI) and/or ultrasonic visualization techniques. MRI is produced by complex interactions of magnetic and radio frequency fields. Materials for enhancing MRI visibility include, but are not limited to, metal particles of gadolinium, iron, cobalt, nickel, dysprosium, dysprosium oxide, platinum, palladium, cobalt based alloys, iron based alloys, stainless steels, or other paramagnetic or ferromagnetic metals, gadolinium salts, gadolinium complexes, gadopentetate dimeglumine, compounds of copper, nickel, manganese, chromium, dysprosium and gadolinium. To enhance the visibility under ultrasonic visualization the stent 18 of the present invention may include ultrasound resonant material, such as but not limited to gold. Other features, which may be included with the stent 18 of the present invention, include radiopaque markers; surface modification for ultrasound, cell growth or therapeutic agent delivery; varying stiffness of the stent or stent components; varying geometry, such as tapering, flaring, bifurcation and the like; varying material; varying geometry of stent components, for example tapered stent filaments; and the like. In any event, stent 18 is an example of a radially self-expanding, i.e. elastically compressible to a reduced-radius, providing an axially-elongated state for positioning within catheter 20 near distal end 26 as shown in FIG. 1. When released from the catheter 20, stent 18 radially self-expands into a substantially conforming surface contact with a body lumen, including vascular and non-vascular lumens, or body tissue (not shown). In some instances the stent may have to be further expanded with a balloon or similar device.

Control device 28 includes a distal tip 30 and a proximal end 32 disposed proximally of catheter proximal end 24. This may allow a physician to manipulate the control device 28, moving it axially relative to catheter 20, usually with the aid of a hub or handle (not illustrated). When stent 18 is radially compressed in the reduced-radius configuration about distal tip 30, the stent 18 is designed to be maintained in a releasable engagement with the control device 28, and thus tends to track or follow axial movement of the control device 28. Accordingly, with the distal end 26 of catheter 20 positioned at the treatment site, the catheter 20 may be withdrawn proximally while the control device or inner member 26 is held in place. Because of its releasable engagement with control device 28, stent 18 likewise tends to stay in place rather than following proximal movement of the outer catheter 20, whereupon stent 18 is progressively released from the outer catheter 20 for radial self-expansion. After complete release and expansion of the stent 18, catheter 20 and control device 28 are proximally withdrawn.

Even after stent 18 is partially released, it should continue to track axial movement of control device 28, so long as the distal region of the stent 18 is radially compressed about distal tip 30. This feature allows the stent 18 to be recaptured or re-constrained after a partial deployment. Should the need arise to reposition a partially deployed stent 18, control device 28 can be held stationary while catheter 20 is moved distally to recapture the stent 18. Alternatively, the catheter 20 can be held stationary while the control device 28 is moved distally to recapture the stent 18. In other words, either of the catheter 20 and/or the control device 28 may be held or moved relative to one and the other. Then, after the assembly is moved to reposition the stent 18 as desired, catheter 20 can be moved in the proximal direction, releasing the stent 18 once again.

System 16 further includes a stent loading component in the form of a stent capturing device 34. At the proximal end of the device 34 is a stent-capturing section, such as stent-engaging member 36, which may function as a distensible stent-engaging member. The stent-engaging member or basket 36 may be formed with multiple helically wound interbraided strands 36′, so that it is similar in construction to stent 18. At the distal end 38 of the stent-engaging member 36, at least some of the strands or filaments 36′ may be gathered to reduce the stent-engaging member diameter and integrally couple the stent-engaging member 36 to an elongate, pliable moving member or stent-engaging member puller 40. The stent-engaging member puller 40 may be formed of a polymer such as polyvinyl chloride (PVC), PEEK, PP, PA or any of the above-described polymers. Other suitable materials for the stent-engaging member puller 40 may include metals such as stainless steel, nitinol and/or any of the above-described metals and/or alloys. Stent-engaging member puller 40 may be constructed to have axial stiffness, but alternatively can be formed as pliable and inextensible in the nature of a metallic coil, chain, or thread, configured to exert sufficient tension to pull stent-engaging member 36 into and through catheter 20 or another stent-confining structure.

FIG. 2 illustrates a band 42 used to couple the stent-engaging member 36 to the puller 40. The band 42 may be a heat shrink tube formed of flexible polyolefin. Alternatively, the stent-engaging member strands 36′ may be embedded into the proximal end 41 of the puller 40, or fused at the puller proximal end 41. The stent-engaging member 36 and puller 40 also may be attached by welding or swaging, or bonded with an adhesive. The strands 36′ of stent-engaging member 36 may be formed of stainless steel or another metal, or a polymer. In general, materials suitable for the strands or filaments of stent 18 also are suitable for the strands 36′ of stent-engaging member 36. Any suitable materials, such as materials used in catheters, may be used to construct the capturing device 34.

Stent-engaging member 36 may be compliant, and may be resilient as well, depending on the material selected for the stent-engaging member strands 36′. The stent-engaging member 36 may be open at its proximal end 44 to receive stent 18 in a relaxed or enlarged-radius state as shown near the stent-engaging member in broken lines. This is generally the shape assumed by stent 18 when it is not subject to any external forces. As compared to its configuration when loaded into catheter 20, stent 18 in the relaxed state has a larger radius and shorter axial length. In any event, the opening at proximal end 44 is large enough to receive stent 18 when in the relaxed state. At the same time, that part of stent-engaging member 36 that surrounds stent 18 is shorter than the axial length of the stent 18, and in some embodiments no more than about half the stent length. In other words, a proximal region of the stent 18 extends beyond the stent-engaging member 36, and may constitute at least one-half of the stent length.

Alternative stents and prostheses may be formed according to non-braided configurations in which the stent, whether in the relaxed state or the reduced-radius state, has substantially the same axial length. Capturing device 34 is suitable for this type of stent as well. Preferably, the stent-engaging member 36 used with a “non-shortening” stent or prosthesis likewise is configured such that its radial reduction does not lead to any substantial axial elongation. Alternatively, a braided stent-engaging member is selected such that, despite any axial elongation accompanying radial contraction, a proximal region of the radially contracted stent remains free of the stent-engaging member.

As seen in FIG. 3, distal tip 30 of the control device 28 supports a stent retaining feature in the form of a holding sleeve 46 surrounding the tip 30. Sleeve 46 may be formed of a lower durometer or soft material. The holding sleeve 46 is sized such that when stent 18 is radially compressed to the reduced-radius state about the sleeve 46, the stent strands are pressed into the sleeve 46 to at least slightly elastically deform the sleeve 46. As a result, the frictional coupling of the stent 18 and sleeve 46 is stronger than any frictional coupling between the stent 18 and outer catheter 20 when it surrounds the stent 18. Thus, when the outer catheter 20 and control device 28 are moved axially relative to one another, stent 18 tends to track the movement of the control device 28 rather than the outer catheter 20.

One purpose of system 16 is to afford a convenient and reliable loading of stent 18 into outer catheter 20 for deployment. In many systems, stents or other prostheses are loaded into their respective delivery catheters well in advance of the anticipated procedure, and provided to the physician in preloaded form.

Although stents formed of stainless steel and other metals are generally well suited for advance loading, stents formed of many polymeric materials and biodegradable materials generally are not, because they are susceptible to a phenomenon known as stress relaxation or “creep”, in which the stent, maintained in its reduced-radius state for any appreciable length of time, tends to loose at least some of its resiliency. While such a stent may radially self-expand upon release from a catheter or other confining structure, its relaxed-state diameter is often less than before the stent was constrained. Further, in many procedures it is necessary or desirable to sterilize the stent or prosthesis just prior to deployment. Sterilization frequently entails exposure of the stent to elevated temperatures, e.g. 40° C. or higher. For prostheses susceptible to creep, these higher temperatures can increase the tendency of stress relaxation.

When a stent subject to the creep phenomenon is maintained in its relaxed or enlarged-radius state until commencement of a deployment procedure, there remains a need to radially compress the stent and maintain it in its reduced-radius state, but only for several minutes. For most materials, this can help to avoid or minimize the creep phenomenon. System 16, by providing for a convenient and reliable loading of a stent into a delivery catheter, may allow the physician to select from a broader range of stent materials. The physician can choose materials with reference to the tissue being treated and the time the stent is expected to remain at the treatment site, with less concern about whether the stent material is suitable for preloading.

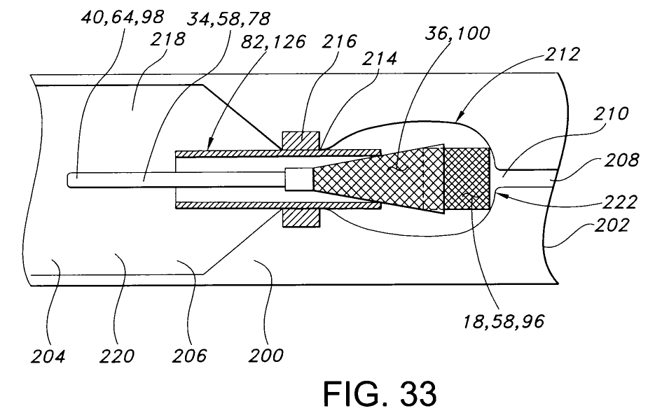

Further, should it be necessary or desirable to sterilize stent 18 during manufacturing, sterilization can be accomplished while the stent remains in its relaxed, enlarged-radius state. The components that come into contact with stent 18 during the procedure may be sterilized as well. Such components, such as capturing device 34, control device 28 and outer catheter 20, may be configured in an assembly or package that also includes the stent, for simultaneous sterilization. A package suitable for this approach is illustrated in FIG. 33 and discussed below.

Accordingly, the utility of a prosthesis susceptible to creep is enhanced when it is maintained in a relaxed, enlarged-radius state until the beginning of a deployment procedure, and further when it is maintained in the relaxed state while being sterilized.

In FIG. 4, system 16 is shown with its components in position for loading stent 18 into catheter 20. More particularly, puller 40 is inserted by its distal end into lumen 22 of the outer catheter, and moved distally through the lumen 22 until stent-engaging member 36 is disposed near proximal end 24, i.e. near a proximal entrance to lumen 22. Stent 18 in its enlarged-radius state is inserted at least partially into stent-engaging member 36 so that the stent-engaging member 36 surrounds a distal region 50 of the stent 18 while a proximal region 48 of the stent 18 remains outside the stent-engaging member 36. Accordingly, stent 18, like the stent-engaging member 36, is disposed near the proximal entrance to the catheter lumen 22. Finally at this stage, stent 18 surrounds distal tip 30 of control device 28. This is conveniently considered a “ready” position for stent loading.

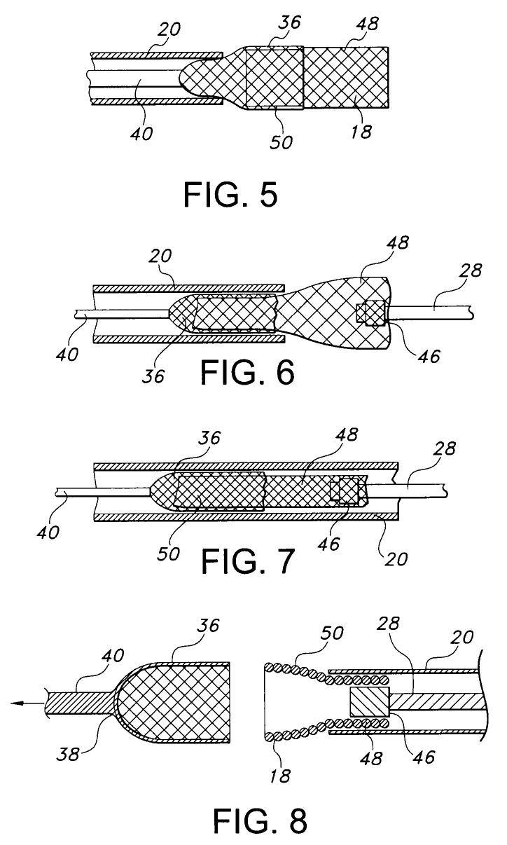

The loading sequence beyond the ready position is shown in FIGS. 5-9. Pulling member 40 has a length sufficient to provide for its extension beyond distal end 26 of outer catheter 20 when in the ready position. Loading proceeds by pulling member 40 distally relative to catheter 20, to draw stent-engaging member 36 distally into lumen 22, further distal movement drawing stent 18 into the lumen 22 as well. Alternatively, catheter 20 could be pushed to the pulling member 40. This progressively radially compresses stent-engaging member 36, and likewise compresses the stent 18 towards its reduced-radius state as seen from FIGS. 5 and 6. With stent-engaging member 36 completely within the lumen 22, member 40 is pulled further in the distal direction to progressively compress stent 18 along proximal region 48, until the stent 18 is compressed over its entire length as shown in FIG. 7. Because the desired alignment of the stent 18 with control device 28 is effected during this stage, the proximal region of the stent is compressed into the reduced-radius state about holding sleeve 46. In this position stent 18 is releasably engaged with holding sleeve 46, and thus will follow axial movement of control device 28 when the device is moved axially relative to the outer catheter.

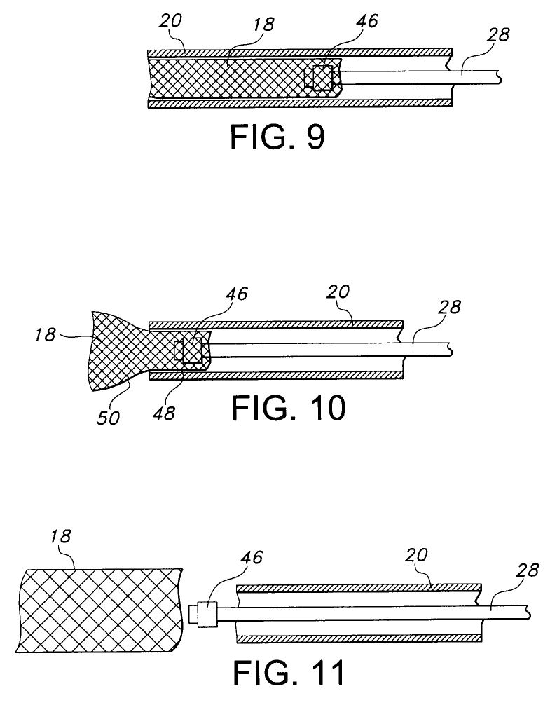

At this point, stent-engaging member pulling member 40 can be pulled distally to draw stent-engaging member 36, stent 18 and control device 28 through lumen 22 until the stent-engaging member 36 and stent 18 reach distal end 26 of the catheter 20. In some embodiments, however, the components may be controllable, individually or in combination. The components are moved further in the distal direction until capturing device 34 is free of the outer catheter 20. At this point the distal region of stent 18 may be disposed outside the catheter 20 and may expand radially as shown in FIG. 8. Proximal region 48 remains inside the catheter 20, constrained in the reduced-radius state about distal tip 30. Accordingly, control device 28, when pulled back (proximally) relative to catheter 20, draws stent 18 back into the catheter 20 as shown in FIG. 9. This essentially completes the loading of stent 18.

With the catheter 20, stent 18 and control device 28 arranged as shown in FIG. 9, they are ready for the stent deployment procedure, which begins by inserting these components into the body of a patient while proximal portions of catheter 20 and control device 28 remain outside the body. The components are moved in a distal direction through a body lumen until distal end 26 or catheter 20 is located near the intended treatment site. A previously inserted guidewire, not shown, may be used to direct the catheter 20 along a desired intraluminal path, and/or the catheter 20 may be equipped with a bendable distal tip to facilitate guidance through the body lumen without the aid of a guidewire.

In either event, with the outer catheter distal end 26 at the treatment site, control device 28 is held in place, while outer catheter 20 is moved in the proximal direction, by manipulating a hub or handle along the proximal portions of these components that remain outside the body. Any of these components or handles may be moved relatively to one and the other, including simultaneous movement. As the catheter 20 is proximally withdrawn, distal region 50 of stent 18 is released from catheter 20 and undergoes radial self-expansion, encountering surrounding tissue of the body lumen as seen in FIG. 10. At this stage, the physician can check the stent position to determine whether it is properly aligned with the intended treatment site. If the stent is properly aligned, proximal withdrawal of catheter 20 may continue until the stent is completely released from the catheter, as seen in FIG. 11.

Returning to FIG. 10, should the physician determine at this stage that the stent is not properly aligned, catheter 20 instead can be moved distally to recapture stent 18. This is possible because of the releasable engagement of stent 18 with control device 28, which can prevent the stent from moving distally with the catheter. Once the stent 18 is recaptured, the physician can re-position the catheter distal end 26 and resume the deployment.

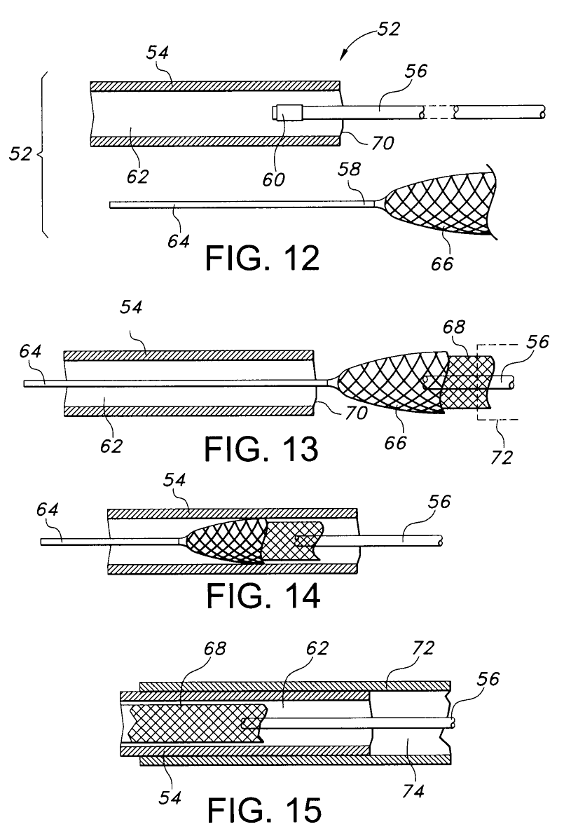

FIG. 12 illustrates an alternative embodiment stent loading system 52 including a tubular loading member 54, a control device 56 which can be similar to control device 28, and a capturing device 58 which can be similar to capturing device 34. Tubular member 54 may be an intermediate or transitional member, in the sense that capturing device 58 is used to load a stent or other prosthesis, when in surrounding relation to a distal tip 60 of the control device as previously described, into loading member 54 rather than into a delivery catheter. After loading, the stent while engaged with control device 56 can be transferred from tubular member 54 into a delivery catheter, whereupon the tubular member can be removed to leave the stent and control device inside the catheter, positioned for deployment.

Tubular loading member 54 can be formed of any suitable polymer, for example, but not limited to, polycarbonate (PC), PA, PTFE or PET. Tubular member 54 can have an outer diameter comparable to an outer diameter of the delivery catheter, although this is not required. A lumen 62 runs through the tubular member, and has an inner diameter greater than the outside diameter of control device 56. In particular, the lumen diameter is selected to maintain a stent in a reduced-radius state about distal tip 60.

Tubular member 54 requires an axial length sufficient only to maintain a stent or other prosthesis in its reduced-radius, axially-elongated state. Consequently, the tubular member 54 is much shorter than the catheter used for intraluminal delivery of the prosthesis, e.g. from about 50 centimeters to about 100 centimeters shorter. These lengths are non-limiting, and the procedure at hand governs the size of the delivery catheter, the prosthesis (and thus the tubular member), and other components. As just noted, capturing device 58 of system 52 can be used to load the stent into tubular member 54 rather than into a delivery catheter. Thus, a pull member 64 of capturing device 58 can be much shorter than puller 40 of capturing device 34, even when stent-engaging members 36 and 66 are substantially the same size.

Capturing device 58 may be used to load a stent 68 into tubular member 54 in much the same manor as capturing device 34 is used to load stent 18 into outer catheter 20. The capturing device 58 is inserted into member 54 by puller 64, and then moved distally until stent-engaging member 66 is located near a proximal end 70 of the tubular member 54 as seen in FIG. 13. Stent 68 (or other prosthesis) is contained by its distal end region in stent-engaging member 66, in its enlarged-radius sate. A proximal region of stent 68 may remain outside stent-engaging member 66 and surrounds distal tip 60. A convenient option afforded by system 52 is to insert control device 56 into the delivery catheter before distal tip 60 is positioned within the stent, whereby the control device over most of its length is contained within a delivery catheter 72 indicated in broken lines at FIG. 13.

From this position, pull member 64 is moved distally or the tubular member 54 is advanced until stent 68 resides entirely within tubular member 54, as shown in FIG. 14. At this point pull member 64 can be moved further in the distal direction, pulling a distal region of stent 68 distally beyond tubular member 54 to completely free the stent-engaging member 60 from the stent 68 and tubular member 54, in the manner previously described in connection with system 16. Next, control device 56 is moved proximally to draw the stent 68 back into the tubular member 54.

At this stage, control device 56 can be moved proximally relative to delivery catheter 72, thus to draw stent 68 and tubular member 54 toward the delivery catheter, positioning a proximal end of the tubular member in confronting relation to a distal end of catheter 72 as shown in FIG. 15. At this stage, with the tubular member abutting the catheter, control device 56 can be pulled proximally to draw stent 68 out of tubular member 54 and into the delivery catheter. When the stent 68 is transferred completely from tubular member 54 into catheter 72, the tubular member can be removed, and the stent is ready to be deployed.

In system 52, the diameter of lumen 62 of tubular loading member 54 preferably is equal to the diameter of a lumen 74 of catheter 72. This better insures that catheter 72, like tubular member 54, can maintain the stent in its reduced-radius state about distal tip 60 so that the stent tends to track axial movement of the control device. If desired, a retaining feature like holding sleeve 46 is mounted on distal tip 60. Further, the delivery catheter can be enlarged at the distal end, to form a socket to receive tubular member 54. Moreover, tubular member 54 can be modified to easily move inside catheter 72. The tubular member 54 may be lubricated, may have ribs or bumps to reduce contact area and/or may include low friction materials or portions.

As compared to system 16, system 52 requires an extra component in the form of tubular member 54. Stent loading with system 52 requires the additional step of drawing the stent from the tubular member to the delivery catheter. Nonetheless, system 52 may be preferred. Loading the stent or other prosthesis into tubular member 54, as compared to loading stent 18 into catheter 20, may be easier and less distracting to the physician, primarily because of the reduced length of capturing device 58 and tubular member 54. While stent deployment with system 52 still requires components of greater axial length, namely catheter 72 and control device 56, the control device can be loaded into the delivery catheter well in advance of the procedure, when time is not critical. Stent loading with the shorter components in system 52 can also reduce the risk of accidental dropping and/or the stent can come preloaded with the catheter 72 to avoid possible contamination of the components. The preloading may by performed by the manufacturer or supplier of the system or may be prepared by a practitioner's assistant prior to use by the practitioner. Thus, system 52 affords a level of convenience that can reduce risk to the patient associated with procedural delays, particularly in time-critical procedures.

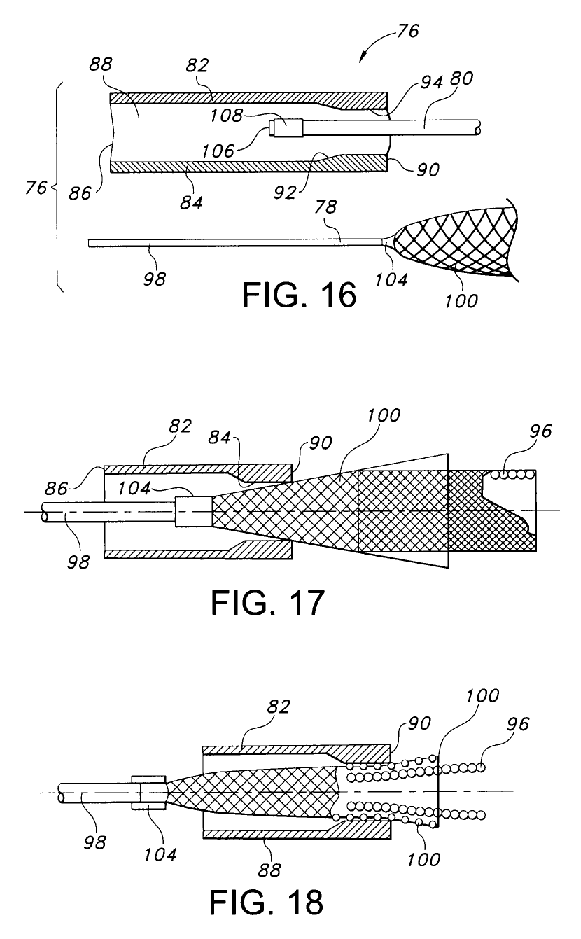

FIG. 16 shows an alternative embodiment prosthesis loading system 76 including a capturing device 78 similar to capturing device 58, a control device 80 similar to control devices 28 and 56, and an intermediate tubular loading capsule 82 having a length similar to that of tubular member 54, for the same size of prosthesis. Capsule 82, like tubular member 54, is constructed of polymeric material. More particularly, capsule 82 may have a wall 84 of uniform thickness over most of its length including the distal end 86, thus to define a lumen 88 with a uniform diameter over most of the capsule length. Near a proximal end 90 of the capsule 82, the thickness of wall 84 may be gradually increased to form an inward incline 92 in the proximal direction and a reduced-diameter neck 94. Like tubular member 54, capsule 82 can function as an intermediate component in the loading process and is removed prior to body insertion. Accordingly, the capsule 82 may be constructed of body compatible material, but need not be.

With reference to FIG. 17, a stent 96 is loaded into capsule 82 in much the same manner as it would be loaded into tubular member 54. Capturing device 78 may be guided through lumen 88 by moving a pull member 98 distally until a stent-engaging member 100 is near proximal end 90. Stent-engaging member 100 can contain a distal region of the stent 96, while a proximal region 102 of the stent 96 may remain outside the stent-engaging member as shown. A band 104 may couple the stent-engaging member 100 to the pull member 98.

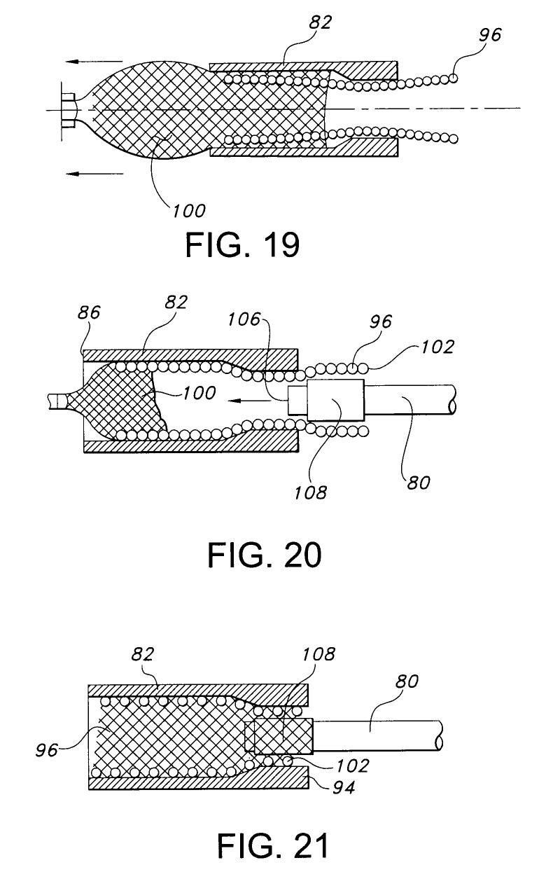

As member 98 is pulled in the distal direction, stent-engaging member 100 and stent 96 may be progressively radially compressed as shown in FIG. 18. Continued distal movement draws stent-engaging member 100 beyond neck 94, leaving proximal region 102 disposed along the neck and partially exposed proximally of capsule 82 as shown in FIG. 19. The larger capsule diameter distally of neck 94 may permit radial expansion of stent-engaging member 100 and stent 96, with further expansion allowed distally of the capsule.

At this stage, distal tip 106 of control device 80 including a retaining sleeve 108 can be moved distally to be surrounded by proximal region 102 of the stent 96, as shown in FIG. 20. Further, advancement of the control device 80 can move the proximal-region 102 of the stent 96 and distal tip 106 into capsule 82 to be surrounded by neck 94 as shown in FIG. 21. Stent-engaging member 100 remains captured between the distal region of stent 96 and capsule 82 at this stage. Consequently, pull member 98 can be used to remove stent-engaging member 100 distally from the capsule, with relative ease and without pulling stent 96 with it. The stent 96 remains loaded in the capsule 82 with its proximal end surrounding distal tip 106 at neck 94, as shown in FIG. 21.

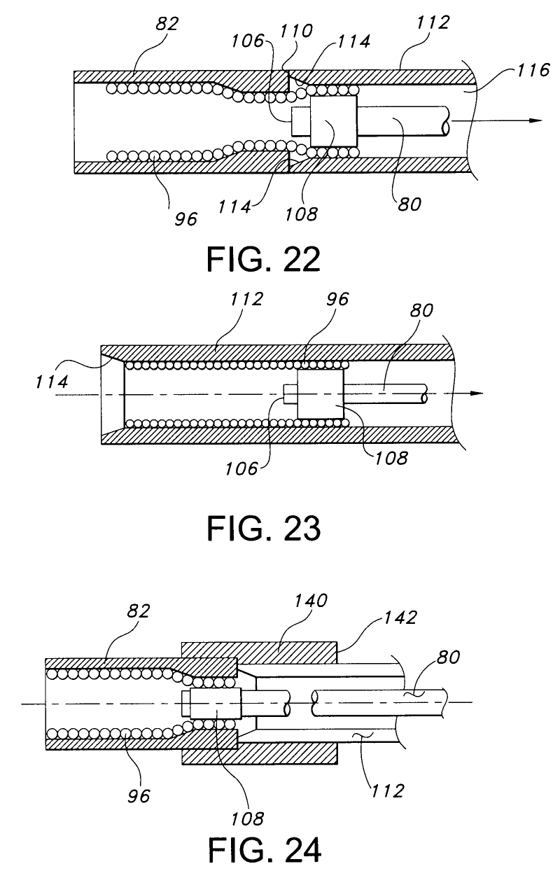

At this stage, proximal end 90 of the capsule and a distal end 110 of a delivery catheter 112 may be placed in confronting, centered relation to one another. Preferably, control device 80 has been preloaded into catheter 112 as described in connection with system 52. With the capsule and catheter maintained in confronting relation, control device 80 can be pulled proximately, which draws stent 96 proximally into the catheter as seen in FIG. 22, since the stent proximal end remains radially compressed and releasably engaged with sleeve 108. Delivery catheter 112 over most of its length has an inside (lumen) diameter about the same as the diameter of neck 94, to maintain the releasable engagement of the stent 96 and control device 80 once they are contained in the catheter rather than the capsule. However, catheter 112 may be tapered near its distal end as indicated at 114, to facilitate the use of control device 80 to draw stent 96 proximally into a lumen 116 of the catheter. Sufficient proximal movement of the control device 80 positions stent 96 along the catheter distal end as shown in FIG. 23, for delivery and deployment. Distal movement of the control device 80 relative to the catheter 112 may release the stent 96 from the catheter 112 and the control device 80.

Loading system 76 provides shorter components for stent loading, thus to afford the advantages discussed above in connection with system 52. Further advantages arise due to the controlled variance in the wall thickness of the capsule. Transfer of the stent to the delivery catheter requires alignment and engagement of the stent and the control device. The thinner wall (larger lumen diameter) along most of the capsule length allows stent 96 to expand to a larger diameter when contained in the capsule. This is particularly important along the distal region of the stent, where stent-engaging member 100 remains captured between the stent and capsule just before its removal. The larger diameter substantially reduces the force necessary to pull stent-engaging member 100 away from the stent and capsule, and minimizes any tendency in stent 96 to follow distal travel of the stent-engaging member. Larger diameters may also reduce plastic deformation of the stent.

Moreover, the confronting relation of the capsule and catheter can eliminate the need to insert the capsule into the catheter when transferring the stent. The capsule walls can have more thickness, and the capsule lumen can be larger in diameter over the majority of the capsule length, necked down near the proximal end to match the diameter of catheter lumen 116.

FIG. 24 illustrates an optional alignment feature for system 76, in the form of a socket 140. A lumen 142 extending through the socket includes a proximal section with a diameter corresponding to the outside diameter of delivery catheter 112, and a larger-diameter distal section corresponding to the outer diameter of loading capsule 82. When a distal region of catheter 112 and a proximal region of capsule 82 are inserted into lumen 142, they can be advanced toward one another until they are engaged in the desired confronting relation, as shown in FIG. 24.

To use socket 140 during loading, catheter 112 can be inserted distally into lumen 142. Then, control device 80 is inserted into lumen 116 to position distal tip 106 proximate the catheter distal end, extending just beyond socket 140. Alternatively, the control device can be inserted into the catheter before inserting the catheter into the socket.

In either event, with stent 96 partially loaded into capsule 82 (see FIG. 20), distal tip 106 is extended distally beyond socket 140 and inserted into proximal region 102, whereupon capturing device 78 can be moved distally to pull the stent into the capsule, thus aligning the distal tip with neck 94 as shown in FIG. 24. Next, the assembly including capsule 82, stent 96 and control device 80 can be moved proximally to insert the capsule into lumen 142, with continued proximal movement bringing the capsule into the desired confronting relation to catheter 112 as shown.

At this stage, control device 80 can be pulled in the proximal direction relative to catheter 112. Stent 96, releasably engaged with the control device, should move proximally with the control device and thereby is transferred from capsule 82 into the catheter. Alignment socket 140 can provide for a more convenient and more reliable transfer of the stent into the delivery catheter while demanding less of the physician's time and attention. The socket 140 accurately centers capsule 82 relative to catheter 112 as the capsule is inserted into lumen 142. Centering is maintained during insertion, until the capsule engages the delivery catheter. Then, as the physician moves control device 80 proximally to draw stent 96 into the catheter, socket 140 is designed to maintain the desired confrontation and alignment.

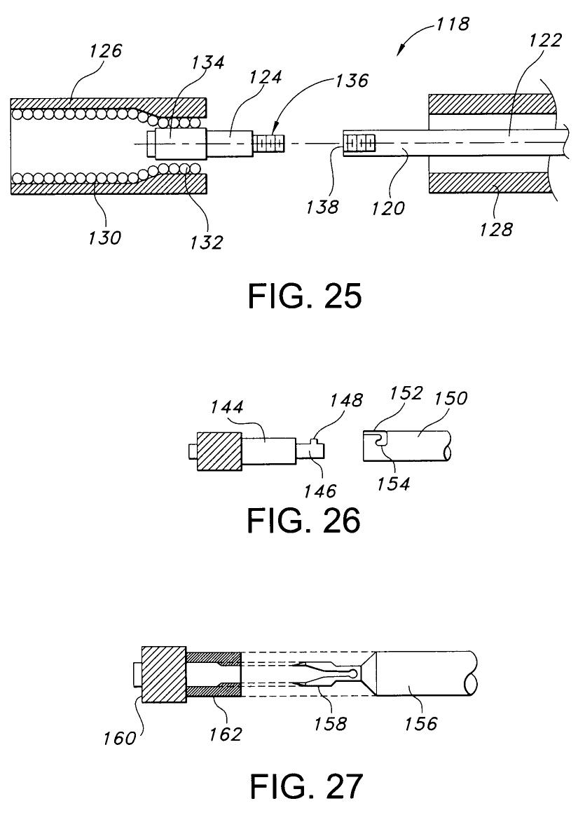

FIG. 25 illustrates in part an alternative embodiment stent loading and deploying system 118 including a control device 120 with an elongate proximal section 122 and a distal tip 124, a loading capsule 126 similar to capsule 82, and a delivery catheter 128. System 118 further may include a capturing device (not shown) similar to capturing devices 58 and 78 for loading a stent 130 into the capsule, with a distal region 132 of the stent surrounding distal tip 124 along a retaining sleeve 134, as shown. In a departure from the other systems, distal tip 124 can be removably mounted to proximal section 122, through a connector 136 with external threads along the distal tip and complementary connector 138 with internal threads formed at the distal end of section 122.

The releasable coupling can provide for a more convenient loading of stent 130 into the capsule, particularly when disposing the distal tip within the proximal region of the stent, and when moving the stent and distal tip into the capsule, as shown in FIGS. 20 and 21 with respect to system 76. Detachable distal tip 124 can be quite short as compared to the length of the control device, as previously described. More generally, the distal tip length is at most about one-tenth the length of the control device. Thus, loading the stent into the capsule can be simplified by avoiding the need to handle the complete control device until after the stent is loaded into the capsule. After stent loading, the assembly including stent 130, capsule 126 and distal tip 124 can be coupled, for example with threads, to section 122 of the control device.

FIGS. 26 and 27 illustrate alternative control device couplings. In FIG. 26, a distal tip 144 includes a reduced diameter proximally extending connector 146 and a pin 148 extending radially outwardly from connector 146. A proximal connector 150 of the control device includes a distal groove 152, shaped to accommodate pin 148 and thus allow a proximal insertion of connector 146. When distal tip 144 is rotated a quarter turn following insertion, pin 148 is captured in a portion 154 of the groove, to maintain distal tip 144 engaged with proximal connector 150 of the control device. In FIG. 27, a proximal connector 156 of a control device can be equipped with a pair of resilient prongs 158 at its distal end. The control device further can include a distal tip 160 incorporating a socket 162 shaped to receive the prongs for a snap fit.

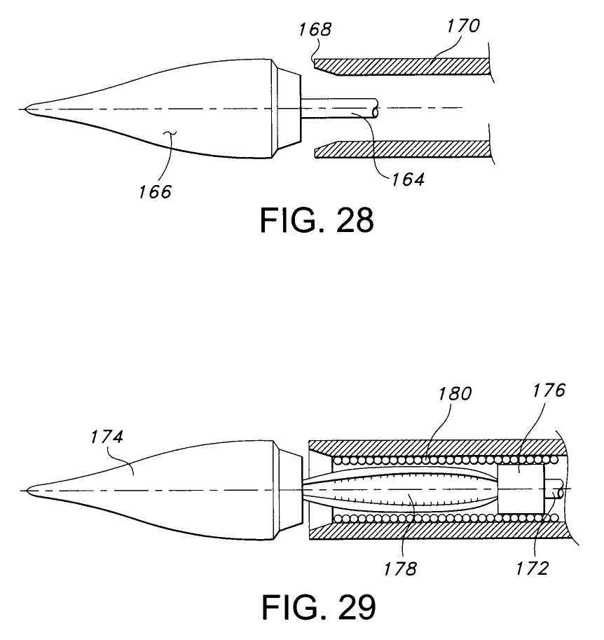

Regardless of the style of coupling, loading of the stent or other prosthesis may be more convenient and less demanding of the physician's attention when the control device has a detachable distal tip. Another advantage of the releasable coupling is that a variety of different distal tips can be connected, alternatively, to the same proximal section of a control device. For example, FIG. 28 illustrates a distal tip 164 incorporating a tapered atraumatic tissue dilating member 166, shown distally spaced apart from a distal end 168 of a delivery catheter 170. At its proximal end, distal tip 164 can incorporate any of the couplings illustrated in FIGS. 25-27.

FIG. 29 illustrates another alternative distal tip 172 of a control device, also equipped with an atraumatic tissue dilating member 174. Distal tip 172 can have a substantial length distally of a retaining sleeve 176, to accommodate a catheter balloon 178. A stent 180 surrounds the distal tip along the length of balloon 178, with a proximal region of the stent surrounding sleeve 176 as in previous embodiments.

In one version of this device, stent 180 is radially self-expanding, in which case the stent is deployed by moving the control device distally to release the stent for radial self-expansion. Balloon 178 is a dilation balloon, inflatable against stent 180 after its release, to press the stent radially outwardly into contact with surrounding tissue for a more secure fixation or to reopen the lumen during placement of the stent. Other dilating mechanisms, such as but not limited to a mechanical cage, may suitably be used as a substitute for balloon 178 or in addition to balloon 178.

Alternatively, stent 180 may be a plastically deformable or balloon-expandable stent. In such cases, the loading and deployment system preferably employs an intermediate component such as capsule 82 or tubular member 54. If desired, the intermediate component can be used to radially compress the stent to a diameter equal to or less than the diameter of the lumen through the delivery catheter. In this case stent 180 may not radially expand against the delivery catheter wall when loaded into the catheter, and requires inflation of balloon 178 for its radial expansion. The stent-engaging member of the capturing tool, and the capsule or other intermediate component, can provide a progressive, controlled radial reduction of the stent to the reduced-radius state. Because of the tendency of plastic deformation, the stent may remain compressed until expanded with the balloon. The confining structure is not needed to maintain a compressive force on the stent. This simplifies transfer of the stent to the delivery catheter.

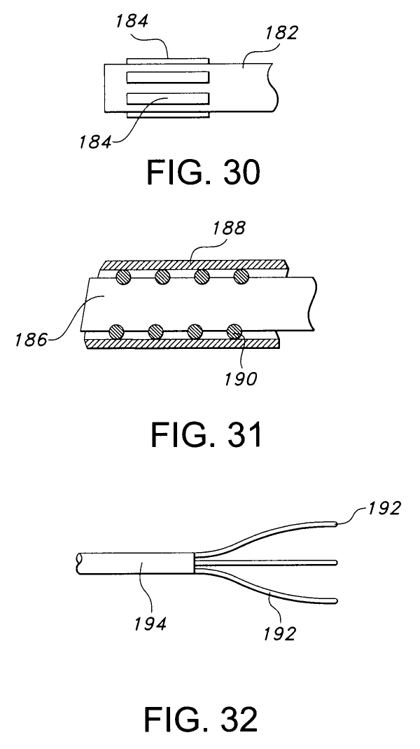

The retaining sleeves described above are one embodiment to providing the releasable engagement of the stent and control device when the stent is in its reduced-radius state. FIG. 30 illustrates an alternative distal tip 182 of a control device in which a plurality of retaining material strips 184 are fixed to the distal tip in lieu of a retaining sleeve. FIG. 31 illustrates another alternative, in the form of a distal tip 186 which can be constructed of a softer or lower durometer material as compared to the material forming a stent confining structure 188, e.g. a delivery catheter, capsule, or tubular member. A stent 190 is shown between the confining member and the distal tip. Because of the softer material, the individual filaments or strands of the stent tend to deform distal tip 186 rather than confining structure 188, thus to become partially embedded in the distal tip. Consequently, when the control device is moved axially relative to the confining structure, stent 190 tends to follow the control device. While this approach eliminates the need for a separate sleeve or other retaining member, the lower durometer material may not provide the desired degree of axial stiffness in the control device. Moreover, the retaining sleeves may have a pattern suitably adapted to the stent design.

While the preferred construction of the capturing device stent-engaging member is an interbraiding of generally helical strands as described, alternative constructions can be deployed here as well, e.g. a stent-engaging member composed of a fabric mesh, or a more tightly woven fabric. In another alternative, strands can be interbraided for the complete length of a capturing device, tightly braided along a “pulling member” section of the device and forming a more open or expanded braid at the end forming the stent-engaging member. In yet another alternative illustrated in FIG. 32, a plurality of elongate strips 192 can be attached to one end of a pulling member 194. The strips can be flexible and self supporting as shown, or linked to one another by transverse cross members which can be elastic if desired. In many of the stent-engaging members, especially the braided and fabric versions, a coating of silicone or other suitable material can be applied to the stent-engaging member interior, to enhance its frictional hold on the stent. This minimizes any tendency of the stent to slip proximally relative to the stent-engaging member as the stent-engaging member and stent are progressively radially compressed. Further, lubrication or low friction material may be disposed or applied to the outside of stent-engaging member to assist, if necessary, its movement through a tube or catheter.

The prosthesis loading and deployment systems of this invention have been described in the context of on-site use by the physician to load a stent or other prosthesis just minutes before that prosthesis is intraluminally delivered and deployed at the treatment site. The loading systems have utility in a variety of other situations as well. For example, where radially self-expanding prostheses (for example when formed of metal) are suitable for preloading and long-term maintenance in the radially-reduced state, the loading systems described herein can be used to preload the prostheses into delivery catheters to provide for faster, simpler and more reliable preloading of these implantable devices.

FIG. 33 is a view, partially in section, of a packaging assembly 200 designed to facilitate the handling of a radially self-expanding stent 18, 58, 96 and components used to load the stent 18, 58, 96 into a delivery catheter 20, 72, 112 or other constraining device 54, 82 at the beginning of a deployment procedure. The assembly 200 can include a container 202 with a profile, for example but not limited to a rectangular profile, when viewed from the top as in FIG. 33. The assembly 200 can include a substantially flat top surface 204, and a recess 206 defining several compartments, including an elongate proximal channel 208 designed to accommodate an inner catheter or other control device (not shown).

At its distal end 210, channel 208 can open to a larger compartment 212 designed to accommodate stent 18, 58, 96 in the relaxed state, a stent-engaging member 36, 100 of a capturing device 34, 58, 78 and the proximal portion of a loading tube or capsule 82, 126. Distally of compartment 212, the recess 206 can be narrowed to provide a neck 214 that accommodates a clamp 216 which may be formed of elastomeric material. Clamp 216 may frictionally engage the loading tube 82, 126 and container 202 to releasably secure tube 82, 126 within the recess 206. Beyond neck 214, capsule 82, 126 may extend distally into a distal compartment 218 designed to accommodate the loading capsule 82, 126 and a pulling member 40, 64, 98 of the capturing device 34, 58, 78.

Stent 18, 58, 96, and to a lesser extent the stent-loading components involved, determine the size of container or tray 202 and the compartments 212, 214, 218 of recess 206. Proximal channel 208 should have a diameter larger than the outer diameter of the control device 28, 56, 80 intended for use with stent 18, 58, 96 to accommodate a distal insertion of the control device distal end 28, 56, 80 into the stent 18, 58, 96. Medial compartment 220 requires a width (vertical dimension in the FIG. 33) sufficient to accommodate the stent 18, 58, 96 and stent-engaging member 36, 100 and further to provide convenient finger access to facilitate handling the stent and stent-engaging member. In addition, a proximal wall 222 of compartment 212 can be positioned to provide a stop that maintains stent 18, 58, 96 within the stent-engaging member 36, 100. Finally, distal compartment 218 may have a length sufficient to accommodate a full distal extension of pulling member 40, 64, 98 with the capturing device 34, 58, 78 loaded as shown. Alternatively, a compliant pull member (not shown) may be formed into one or more loops or otherwise confined into a shorter distal compartment if desired.

Packaging assembly 200 can facilitate stent loading by maintaining the stent 18, 58, 96 and certain loading components in place, allowing the physician to direct his or her attention to other concerns, e.g. proper alignment of an inner catheter or other control device when pulling the stent 18, 58, 96 into the loading capsule 82, 126. Using channel 208 to guide distal travel of a control device 28, 56, 80, the physician can align the distal tip of the control device within the stent 18, 58, 96. Then, while moving pulling member distally to draw the stent-engaging member and stent 18, 58, 96 into loading capsule, the physician can move the control device 28, 56, 80 distally to maintain the desired distal tip position, using channel 208 as a guide. Thus, the capturing device 34, 58, 78, stent 18, 58, 96 and control device 28, 56, 80 can be moved distally in concert, while clamp 216 grips loading capsule 82, 126 to maintain its location relative to tray 202, until the stent is completely contained within the capsule 82, 126, in its reduced-radius state.

Then, with clamp 216 continuing to maintain capsule 82, 126, the physician can continue to move pulling member 40, 64, 98 and the control device distally in concert, until stent-engaging member 36, 100 is free of the capsule 82, 126, then can move the control device 28, 56, 80 proximally to draw stent 18, 58, 96 back into capsule 82, 126 as previously described.

Thus, a salient advantage of the packaging assembly 200 is that it can facilitate an accurate alignment of the control device 28, 56, 80 with the stent 18, 58, 96 during use of the capturing device 34, 58, 78 to draw the stent 18, 58, 96 distally into the loading capsule 82, 126. A further advantage of the package assembly is that it can facilitate sterilization of the stent 18, 58, 96 and loading components. According to one approach, the entire packaging assembly as shown in FIG. 33 is sterilized, then combined with a sterilized inner catheter or other control device 28, 56, 80 to load the stent 18, 58, 96 into the capsule 82, 126. In an alternative approach, the stent 18, 58, 96 is loaded into capsule 82, 126 then sterilized along with the capsule 82, 126 and the control device 28, 56, 80. This approach permits the removal of capturing device 34, 58, 78 before sterilization.

Thus in accordance with the present invention, a variety of prosthesis loading and deploying systems are provided. These systems facilitate on-site loading of prostheses just prior to delivery and deployment procedures, and thus allow the physician to use prostheses that are well suited to the procedure, but not necessarily suited for remaining constrained in a reduced-radius state for extended periods of time. All of the systems advantageously employ control devices that are operable to move a prosthesis in either axial direction when the prosthesis is constrained about the control device in a reduced-radius state. Some of the systems use capsules, tubular members or other intermediate components that are shorter and therefore easier to handle than delivery catheters. Other systems employ control devices with detachable distal tips. The result is a more convenient and simplified loading of stents and other prostheses, allowing physicians to direct their attention more appropriately to the procedure at hand.

Moreover, any of the above-described tubular components which are disposable within the outer catheter tube may be splittable, for example be able to be pulled back out of the catheter in a banana-peel like manner. Furthermore, any of the above-described prosthesis or stent retaining sleeves may them selves be expandable when removed from the catheter. Still furthermore, any the components of the invention may be packaged separately or in any combination.

While various embodiments of the present invention are specifically illustrated and/or described herein, it will be appreciated that modifications and variations of the present invention may be effected by those skilled in the art without departing from the spirit and intended scope of the invention. Further, any of the embodiments or aspects of the invention as described in the claims or in the specification may be used with one and another without limitation.

What is claimed is:

1. An apparatus for loading a radially expandable prosthesis into a prosthesis confining structure for maintaining the prosthesis in a reduced-radius state, comprising:
a prosthesis capturing device with a proximal capturing section that forms a compliant enclosure open at a proximal end to allow an insertion of a radially expandable prosthesis in an enlarged-radius state into the enclosure, to be surrounded by the enclosure over a distal region of the prosthesis;
said capturing device further comprising:
an elongate enclosure moving section, insertable into and moveable distally along a passage of a prosthesis confining structure to locate the enclosure, and thereby also locate a radially expandable prosthesis so surrounded by the enclosure, adjacent a proximal entrance of the passage; and
an elongate control device having a distal end region insertable into a radially expandable prosthesis in the enlarged-radius state;
wherein the control device distal end region is adapted for a releasable engagement with a radially expandable prosthesis when surrounded by the prosthesis in a compressed state, in which the prosthesis tends to track axial movement of the control device; and
wherein said enclosure moving section, with the enclosure and a radially expandable prosthesis so located and with the prosthesis surrounding the distal end region of the control device, is movable distally to draw the enclosure and the prosthesis into the passage to cause a progressive radial compression of the enclosure and prosthesis, radially contracting the prosthesis to the compressed state about the distal end region to effect said releasable engagement.

2. The apparatus of claim 1 wherein the enclosure has an axial length corresponding to an axial length of a radially expandable prosthesis selected for insertion into the enclosure, whereby a proximal region of the prosthesis remains outside the enclosure following said insertion.

3. The apparatus of claim 2 wherein said proximal region of the prosthesis constitutes at least one-half of the prosthesis length.

4. The apparatus of claim 2 wherein the enclosure moving section is operable to pull the proximal capturing section distally through a passage of a prosthesis confining structure until the proximal capturing section exits the passage, leaving the proximal region of the prosthesis in the passage and releasably engaged with the control device.

5. The apparatus of claim 1 wherein the proximal capturing section comprises a stent-engaging member.

6. The apparatus of claim 1 wherein the proximal capturing section comprises a plurality of helically wound and interbraided strands, whereby the capturing section elongates axially as it radially contracts.

7. The apparatus of claim 1 wherein the capturing section comprises a fabric mesh.

8. The apparatus of claim 1 further including a coating applied to an interior portion of the proximal capturing section to enhance friction.

9. The apparatus of claim 1 further including a coating applied to an exterior portion of the proximal capturing section to reduce friction.

10. The apparatus of claim 1 further including a prosthesis retaining feature disposed along the distal end region of the control device.

11. The apparatus of claim 1 further including a prosthesis in the form of a radially self-expanding stent.

12. The apparatus of claim 1 further including a catheter balloon disposed along the control device.

13. The apparatus of claim 10 wherein the retaining feature comprises a holding sleeve surrounding the control device.

14. The apparatus of claim 1 further including a prosthesis confining structure.

15. The apparatus of claim 14 wherein the confining structure comprises a prosthesis delivery catheter.

16. The apparatus of claim 14 wherein the confining structure comprises a tubular loading member having an axial length exceeding an axial length of the prosthesis.

17. The apparatus of claim 14 wherein the confining structure comprises a tubular loading member having a diameter greater than a diameter of the control device and positionable against a delivery catheter selected for delivering the prosthesis to a treatment site.

18. The apparatus of claim 14 wherein the confining structure comprises a loading capsule incorporating a lumen extending axially therethrough.

19. The apparatus of claim 18 wherein the lumen near a proximal end of the capsule is substantially equal to a diameter of a lumen of a delivery catheter selected for delivering a prosthesis to a treatment site, to facilitate a transfer of the prosthesis proximally from the capsule into the selected catheter with a proximal end of the capsule in confronting relation to a distal end of the selected catheter.

20. The apparatus of claim 18 wherein the lumen of the capsule is enlarged in the distal direction.

21. The apparatus of claim 18 further including a socket positionable in surrounding relation to the capsule.

22. The apparatus of claim 1 wherein said elongate control device includes a proximal section removably attached to the distal end region.

23. A stent loading and deploying device, comprising:
a stent confining device for maintaining a radially expandable stent in a compressed state suitable for delivering the stent to an intraluminal treatment site;
a stent capturing device including a proximal capturing section forming a compliant enclosure open at a proximal end to allow insertion of the stent, when in a relaxed state, into the enclosure such that the enclosure surrounds a distal region of the stent;
the capturing device further comprising:
an elongate enclosure moving section insertable into and moveable distally through a passage running axially through the stent confining device to locate the enclosure and the stent so contained in the enclosure adjacent a proximal entrance of the passage; and
an elongate stent control device having a distal end region insertable into the stent when the stent is in the relaxed state;
wherein the distal end region of the control device is adapted for a releasable engagement with the stent when surrounded by the stent and with the stent in the compressed state, whereby the stent tends to follow axial movement of the stent control device; and
wherein the enclosure moving section of the capturing device, with the enclosure and the stent so located and with the stent surrounding the distal end region, is moveable distally to draw the enclosure and the stent into the passage to progressively radially compresses the enclosure and stent, thereby radially contracting the stent to the compressed state about the distal end region to effect said releasable engagement.

24. A process for loading a radially expandable stent into a confining structure for maintaining the stent in a reduced-radius state, comprising:
loading a radially expandable stent, in an enlarged-radius state, into a enclosure such that the enclosure surrounds a distal region of the stent;
providing an elongate control device having a distal end region;
inserting the control device into the stent to position a proximal region of the stent in a surrounding relation to the control device distal end region;
with the stent so loaded and maintained in said surrounding relation to the distal end region, drawing the enclosure and the stent loaded therein distally into a lumen of a stent confining structure to cause a progressive radial contraction of the enclosure and stent as they enter the lumen to contract the stent to a reduced-radius state about the control device distal end region, thereby to effect a releasable engagement of the stent with the control device whereby the stent tends to track axial movement of the control device.

25. The process of claim 24 further comprising providing a prosthesis retaining feature along the control device distal end region.

26. The process of claim 25 wherein said inserting the control device into the stent comprises aligning the retaining feature with a proximal region of the stent.

27. The process of claim 24 further comprising after said drawing of the enclosure and stent,
moving the enclosure and stent distally through the lumen until the enclosure is free of the lumen, leaving the proximal region of the prosthesis in the lumen in said releasable engagement;
removing the enclosure from the confining structure; and
with the enclosure so removed, pulling the control device proximally to draw the distal region of the stent back into the lumen.

28. The process of claim 27 further comprising after drawing the distal region of the stent back into the lumen,
guiding the confining structure, the stent and control device contained therein intraluminally to position a distal end of the confining structure near a selected treatment site; and
with the distal end of the confining structure so positioned, moving the confining structure proximally relative to the control device to release the stent.

29. The process of claim 28 where the control device is substantially fixed during the release of the stent.

30. The process of claim 27 further comprising after drawing the distal region of the stent back into the lumen, inserting the confining structure with the stent and control device contained therein into a delivery catheter to locate the confining structure near a distal end of the catheter; and after locating the confining structure, pulling the confining structure distally while maintaining the position of the control device and stent relative to the catheter, to remove the confining structure while the stent and the control device remain within the catheter.

31. The process of claim 27 further comprising after removal of the enclosure, inserting the control device into a delivery catheter, and moving the control device and confining structure proximally until the confining structure confronts a distal end of the delivery catheter;
and with the confining structure and catheter so confronting one another, pulling the control device proximally to transfer the prosthesis from the confining structure into the catheter.

32. The process of claim 24 further comprising providing an elongate moving member integral with the enclosure and extended distally away from the enclosure, wherein said drawing of the enclosure and stent comprises inserting the moving member into the lumen at a proximal entrance thereof, translating the moving member distally through the lumen to position the enclosure and stent adjacent said proximal entrance, then pulling the moving member distally to pull the enclosure and stent into the lumen.

33. The process of claim 21 further comprising providing a balloon along the distal end region of the control device.

34. An apparatus for guiding a radially expandable prosthesis when in a reduced-radius state, comprising:
a control tip adapted for forming a releasable engagement with a radially expandable prosthesis when surrounded by the prosthesis in a reduced-radius state, in which engagement the prosthesis tends to track axial movement of the control tip;
a first connecting element disposed at a proximal end of the control tip; an elongate, pliable and axially stable control member; and
a second connecting element disposed at a distal end of the control member and adapted for a releasable coupling with the first connecting element to releasably couple the control tip to the control member with the control tip extending distally away from the control member;
wherein the radially expandable prosthesis tends to track the axial movement of the control member when in said releasable engagement with the control tip, and when the control tip and the control member are so releasably coupled.

35. The apparatus of claim 34 further comprising a prosthesis confining structure having a lumen extended therethrough for containing a radially expandable prosthesis in a reduced-radius state;
wherein the prosthesis is adapted to undergo a progressive radial compression from an enlarged-radius state to the reduced-radius state about the control tip as the prosthesis and the control tip are inserted into the lumen.

36. The apparatus of claim 35 further comprising a prosthesis capturing device with a proximal capturing section that forms a enclosure open at a proximal end to allow an insertion of the radially expandable prosthesis in the enlarged-radius state into the enclosure, to be surrounded by the enclosure over a distal region of the prosthesis, said capturing device further including an elongate enclosure moving section coupled to the proximal capturing section;
wherein the enclosure moving section, with the enclosure and the radially expandable prosthesis surrounded by the enclosure and located adjacent a proximal entrance of the lumen, is movable distally to draw the enclosure and the prosthesis into the lumen to cause a progressive radial compression of the enclosure and prosthesis, radially contracting the prosthesis to the reduced-radius state about the control tip to effect said releasable engagement.
